(19) United States
(12) Patent Application Publication
(10) Pub. No.: US 2003/0032864 A1
(43) Pub. Date: Feb. 13, 2003

(54) Laryngoscope

(76) Inventor: John H.P. Friesen, Manitoba (CA)

Correspondence Address: Ade & Company, 1700 360 Main Street, Winnipeg Manitoba, R3C 3Z3, CA

(21) Appl. No.: 10/220,629

(22) PCT Filed: Feb. 16, 2001
(86) PCT No.: PCT/CA01/00196

(51) Int. Cl. A61B 1/267

(52) U.S. Cl. 600/194; 600/190

(57) ABSTRACT

A laryngoscope has a blade mounted on a bracket and handle for manipulation by the doctor. The blade is generally concave with an inner surface for engaging and compressing the tongue of the patient onto the lower jaw of the patient, a tip portion remote from the bracket for engaging the hyoid bone of the patient to effect moving of the epiglottis and an outer surface over which the doctor obtains a direct line of sight through the mouth past the moved epiglottis to the opening through the larynx to the trachea. The blade is substantially rigid with an apex at a position thereon which in use is located adjacent the tongue of the patient. A portion of the blade from the apex to the tip portion is substantially straight and has a length which is adjustable by a manually operable slide.

This invention relates to a laryngoscope which is shaped and arranged to improve direct vision of the opening to the larynx of the patient while increasing the area available for accommodating the tongue of the patient.

### BACKGROUND OF THE INVENTION

Chapter 20 in airway management (“Airway Management: Principles and Practice by Jonathan L. Benumof (1996)”) relates to a number of arrangements of laryngoscope blades. The present invention is concerned with blades of this type which allow direct vision of the doctor across an outer surface of the generally concave blade shape for a position adjacent the upper teeth of the patient over the tongue of the patient to the epiglottis and the opening in the larynx to the trachea.

In order to obtain this direct line of sight, it is necessary to compress the tongue of the patient into the space behind the mandible of the patient and the curvature of the blade is shaped so that the tongue can be compressed while a tip of the blade engages the hyoid bone of the patient. With the device in place, the device can be pulled in a direction relative to the patient upwardly and toward the tongue to effect compression of the tongue and to effect lifting of the hyoid bone and the epiglottis of the patient thus allowing direct vision of the opening in the larynx.

Development in blades of this type include a light channel which runs along the blade at one side of the blade to allow light to illuminate the larynx. In addition, the blade is generally Z-shaped so that one side of the blade has a portion which is raised above the outer surface to allow an increased area for the tongue of the patient. The blade has a width so that the compressed portion of the tongue is sufficiently wide to allow an intubation tube to be inserted over the blade along the line of sight to the opening in the larynx.

A paper on page 262 by Marks, Hancock and Charters of the Canadian Journal of Anaesthesia, Volume 40, No.3, March 1993 analyses the shape of the blade and makes a number of proposals.

Further details are disclosed in the following US patents:

U.S. Pat. No. 4,314,551 of Kadell issued Feb. 9, 1982;

U.S. Pat. No. 5,584,795 of Valenti issued Dec. 17, 1996;

U.S. Pat. No. 5,178,132 of Mahesky issued Jan. 12, 1993;

U.S. Pat. No. 4,573,451 of Bauman issued Mar. 4, 1986;

U.S. Pat. No. 5,036,835 of Filli issued Aug. 6, 1991;

U.S. Pat. No. 4,295,465 of Racz issued Oct. 20, 1981;

U.S. Pat. No. 5,406,941 of Roberts issued Apr. 18, 1995;

U.S. Pat. No. 5,381,787 of Bullard issued Jan. 17, 1995;

U.S. Pat. No. 4,360,008 of Corazelli issued Nov. 23, 1082;

U.S. Pat. No. 4,384,570 of Roberts issued May 24, 1983;

and in PCT application 97/30626 of Abramowitz published Aug. 28, 1997.

However, it is believed that the shape of the laryngoscope to date has not been optimised so there is still opportunity for design of a laryngoscope which maximizes the area allowing compression of the tongue while providing the best line of sight for the doctor.

### SUMMARY OF THE INVENTION

It is one object of the present invention, therefore, to provide an improved laryngoscope which is shaped and adjustable to maximize the space available to receive the tongue of the patient and provide the best direct line of sight of the doctor to the opening in the larynx

According to a first aspect of the invention there is provided a laryngoscope comprising:

a bracket for mounting on a handle for manipulation by the doctor;

a blade attached to the bracket for insertion through the mouth of the patient into the throat of the patient;

the blade being generally concave with an inner surface for engaging and compressing the tongue of the patient onto the space behind the mandible of the patient, a tip portion remote from the bracket for engaging the hyoid bone of the patient to effect moving of the epiglottis and an outer surface over which the doctor obtains a direct line of sight through the mouth past the moved epiglottis to the opening through the larynx to the trachea;

the blade having a thickness between the inner and outer surfaces arranged such that the blade is substantially rigid;

the blade being shaped to define an apex at a position thereon which in use is located adjacent the tongue of the patient;

a portion of the blade from the apex to the tip portion being substantially straight;

a length of the straight portion of the blade from the apex to the tip portion being adjustable.

Preferably the length is adjustable by a manually operable element exposed beyond the mouth of the patient.

Preferably the length is adjustable by a manually operable push slide.

Preferably the push slide passes through a slide channel in the blade.

Preferably the blade includes a light channel for communicating light to illuminate the area adjacent the tip portion and wherein the slide channel is alongside the light channel.

Preferably the slide channel is between the light channel and the blade.

Preferably the tip portion is bent at an angle to the straight portion in a direction toward the inner surface.

Preferably the blade includes a first portion between the apex and the bracket which is arranged at an angle to the straight portion.

Preferably the inner surface of the first portion is substantially straight or it has a slight concave curvature. This piece does not affect the line of sight and hence its shape is of less importance.

Preferably the first portion has a length which is approximately 30% to 45% of the length from the apex to the end of the tip portion in the extended position of the straight portion. In a particularly preferred arrangement, the length of the first portion is of the order or 4 cms and the length from the apex to the tip portion in the extended position is of the order of 10 cms.

Preferably the apex has a radius of curvature which is sufficiently short such that the line of sight of the doctor intersects the apex at the same point regardless of movements of the blade in the mouth which cause changes of angle of the straight portion relative to the oral cavity.

Preferably the apex defines an angle of the order of 120 to 140 degrees. In order to provided devices suitable for different persons, the angle may be adjustable between 120 and 140 degrees; or more preferably three different devices each having a fixed angle of 120, 130 and 140 degrees respectively can be provided for selection by the doctor in response to an assessment of the patient.

Preferably the blade is generally Z-shaped in cross section forming a raised portion alongside the outer surface for receiving a portion of the tongue of the patient.

Preferably the length is adjustable by a manually operable push slide which passes through a slide channel in the blade at a web in the Z-shape.

Preferably the laryngoscope is used in a method of inserting into a patient comprising:

inserting the laryngoscope into the mouth of the patient to a position in which the apex is located over the tongue of the patient;

moving the blade to compress the tongue of the patient;

adjusting the position of the apex relative to the tongue to achieve maximum space to receive the tongue;

and with the apex so positioned, adjusting the length of the straight portion to provide contact between the tip portion and the hyoid bone of the patient.

### BRIEF DESCRIPTION OF THE DRAWINGS

One embodiment of the invention will now be described in conjunction with the accompanying drawings in which:

FIG. 1 is a side elevational view of a laryngoscope according to the present invention.

FIG. 2 is a cross sectional view along the lines 4-4 of the laryngoscope of FIG. 1.

FIG. 3 is a cross sectional view along the lines 3-3 of the laryngoscope of FIG. 1.

FIG. 4 is a cross sectional view along the lines 2-2 of the laryngoscope of FIG. 1.

FIG. 5 is a side elevational view of the laryngoscope of FIG. 1 shown schematically in use.

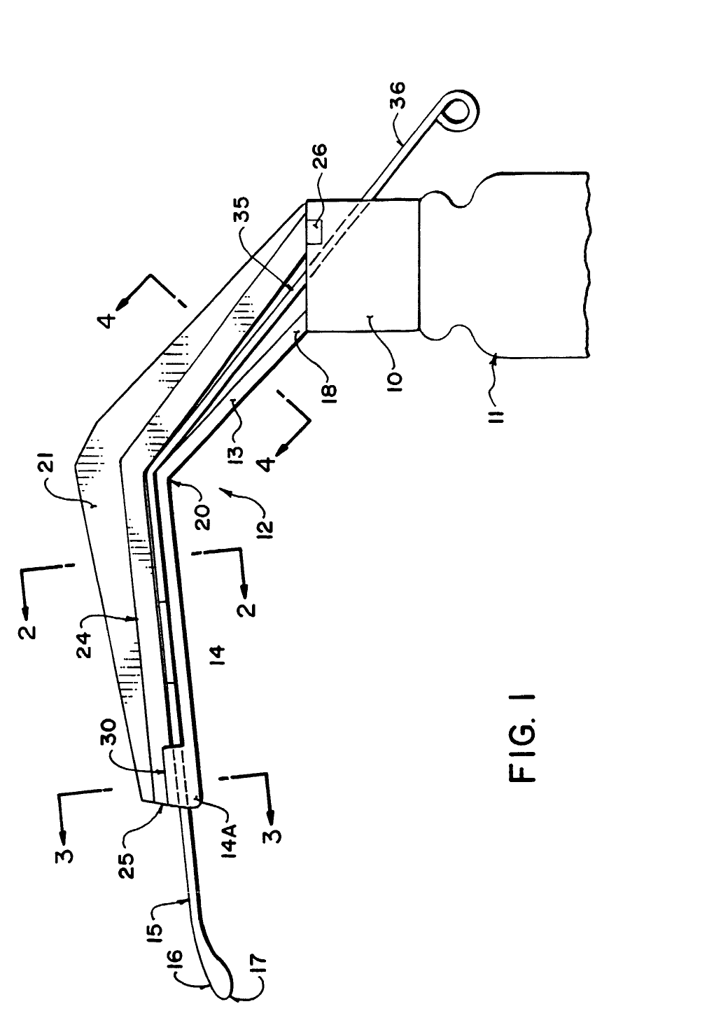

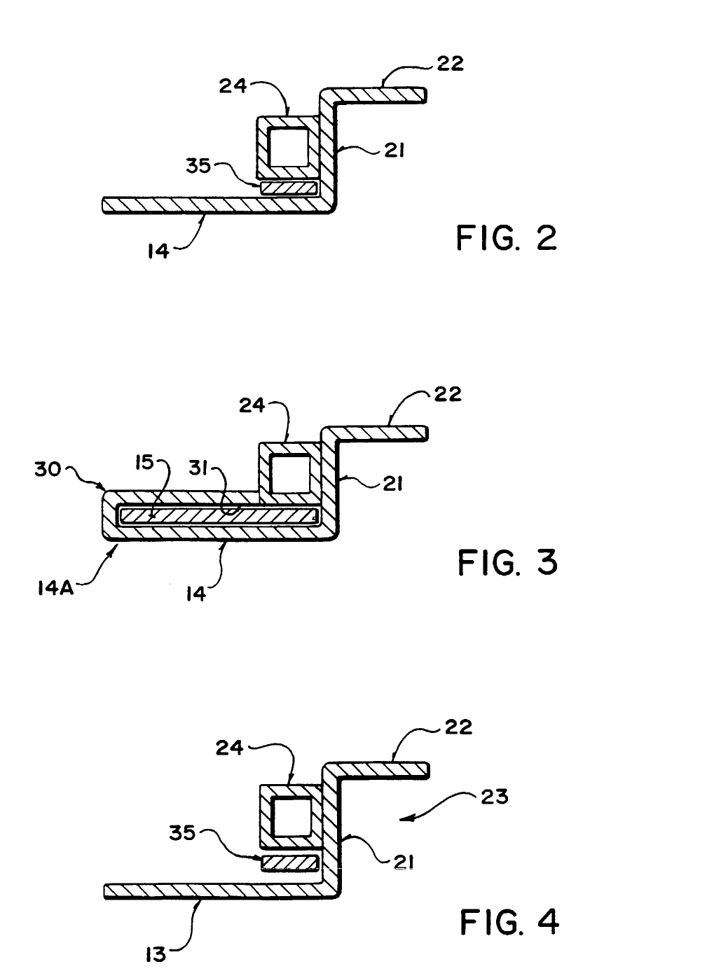

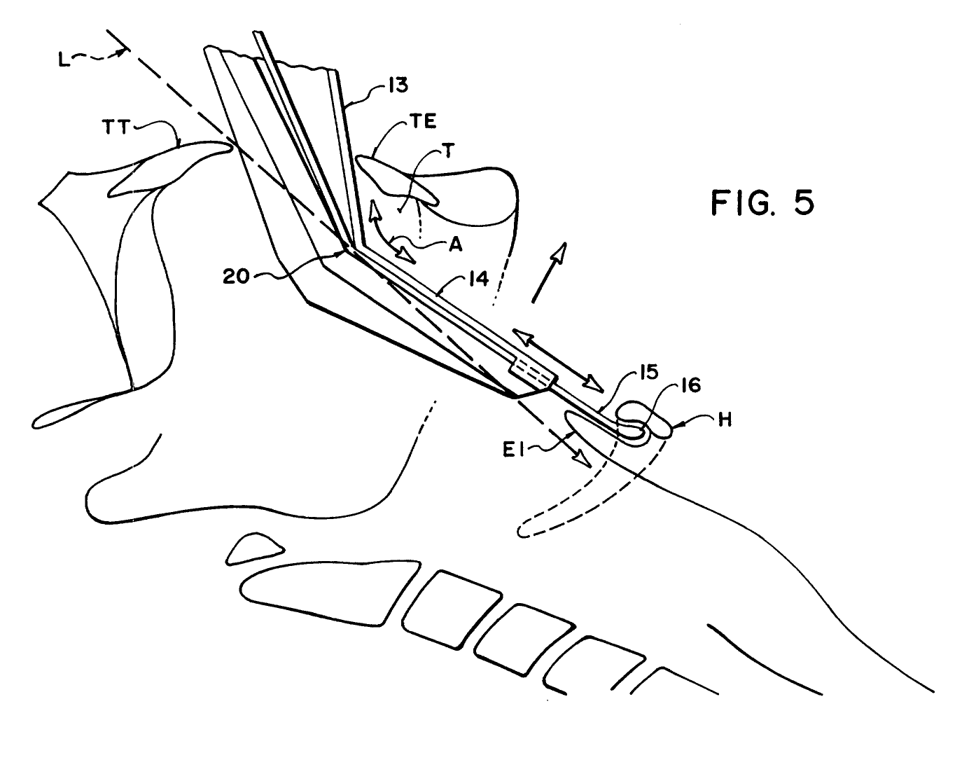

### DETAILED DESCRIPTION

A blade 12 of the laryngoscope is shown in the figures and includes a bracket 10 for attachment to an elongate handle 11 by which the laryngoscope is manipulated by the doctor. The bracket carries rigidly attached thereto the blade 12 which has a first blade portion 13, a second straight blade portion 14, an extension portion 15 forming an extension of the straight portion 14 and a tip portion 16.

The blade is generally concave from a forward most tip 17 through to a base 18 thus forming a concave surface on one side of the blade and a convex or upper surface on the other side. The term “concave” herein is not intended to imply that the blade is smoothly curved since it will be appreciated that the blade shape includes straight portions and an apex 20.

On one side of the blade is formed an upstanding web 21 and a flange portion 22 generally parallel to the blade and spaced outwardly from the blade to define an area 23 into which the tongue of the patient can partly be received.

Alongside the web 21 is provided a light guide 24 which extends to a mouth 25 at a forward end for sending a beam of light along the outer surface portion 15 to illuminate the area of the tip 16. The light guide 24 communicates with a source 26 of light schematically indicated in the bracket 10. In many cases the light source is a light bulb at the bracket which communicates through fiber optic system to the tip 25. However the light guide may simply form an electrical connection to a bulb mounted at the tip 25.

At the end portion 14A of the straight portion 14 is formed a guide 30 which defines a slot 31 into which the blade portion 15 is inserted. This allows the blade portion to slide in a direction longitudinal of the straight portion 14 while maintaining the portion 15 and the portion 14 generally in a common plane. The extension portion 15 is mounted on top of the blade portion 14 and underneath the guide 30. The guide 30 has a length along the length of the blade which is sufficient to prevent twisting or lifting of the extension portion 15 so that it is maintained in its direction coplanar with the portion 14.

Movement of the extension portion 15 in its longitudinal direction is effected by a push rod 35 provided within a recess underneath the light guide 24. The push rod 35 is relatively narrow so that it is in effect hidden underneath the light guide 24 and extends at a band between the light guide and the upper surface of the blade to a handle portion 36 exposed beyond the bracket 10. The handle portion 36 can be thus pushed or pulled to effect adjustment of the position of the adjustment portion 15 relative to the blade portion 14.

The section of the blade thus formed by the portion 14 and the extension portion 15 is thus straight through to the tip portion 16. The tip portion may be curved toward the underside or may simply terminate in a flat straight tip portion forming in effect simply an end of the portion 15.

The blade section as defined by the portions 14 and 15 is formed of a thickness and a material so that it is rigid and is resistant to bending upwardly or downwardly or twisting within the guide in response to the level of forces necessary for actuation of the laryngoscope in use.

The section defined by the portions 14 and 15 extends to the apex 20 at which there is provided a sharp change of direction into the first portion 13.

The first portion 13 has an inner surface which also is straight or having a moderately concave curvature so that there is an angle between the inner surfaces of the portions 13 and 14 defined at the apex which is at the order of 120 to 140 degrees. Without altering the general relationships of the tip 17, the apex 20 and the base 18, the portion 13 could also have a slightly concave curvature.

The apex is relatively of short curvature so that it is relatively sharp. The curvature is sufficiently short that the line of sight of the doctor intersects the apex at a substantially fixed point regardless of changes of angle of the blade within the mouth of the patient. It will be appreciated that a smooth slow curvature causes the line of sight to intersect at different points depending upon the location and angle of the blade as in the conventional prior art. This is disadvantageous since it changes the shape of the blade relative to the oral cavity and prevents the blade from being located properly to maximize the area underneath the blade for receiving the tongue.

In a particularly preferred embodiment, the length of the portion 13 is about 4 cms and the length of the portions 14 and 15 when fully extended is about 10 cms. Thus the apex is located at a position which is approximately 40% of the length along the blade from the base 18 to the tip 17 when in the extended position.

It will be appreciated that the length of adjustment is relatively small since most patients fall generally within a certain range of dimensions so that the adjustment may be only of the order of 2 cms or 20 percent. The blade may be supplied in more than one size in order to accommodate a wider range of sizes of patients.

The Z-shaped cross section of the blade terminates at the end of the portion 14 so that the portion 15 is simply flat or it may have slightly upturned side edge so as to provide additional strength to prevent bending in operation.

In operation, the doctor makes an initial assessment of the required length of the extension portion 15 so that the tip 16 is intended to engage the hyoid bone H of the patient at the epiglottis of the patient. However this assessment is of course initially approximate and cannot be directly measured at this time due to the necessity for rapid insertion of the intubation tube.

With the blade initially adjusted, the blade is inserted through the mouth into the into the oral cavity including the throat so that the apex 20 reaches a position behind the bottom teeth TE of the patient and over the tongue T of the patient. The doctor makes an adjustment of the position of the blade in the direction of the arrow A so as to move the apex 22 to the best position to maximize the area and allow the maximum area for the tongue to be received. This adjustment is effected by the doctor moving the apex 20 slightly forwardly and backwardly along the arrow A until the blade can be moved upwardly compressing the tongue to its best position.

With the apex 20 in the best position for compression, the doctor effects adjustment of the length of the extension portion by operating the handle 36 externally of the bracket so that the tip 16 is detected to directly move into position in contact with the hyoid bone.

In this position after adjustment, the doctor is ensured of the best possible line of sight L which passes under the top teeth TT of the patient and intersects the apex 20 and provides viewing of the area under the moved epiglottis El. This allows the direct line of sight to the opening in the larynx.

The relatively sharp apex ensures that the line of sight always intersects at the same position at the apex. The apex can be positioned to best obtain compression of the tongue and the tip can be adjusted by increasing or decreasing the length of the extension portion so that it engages the hyoid bone when the apex is at its best position.

The device as shown is fixed at the apex 20. It is preferred that further adjustability of the device is provided by supplying blades with different angles of for example 120, 130 and 140 degrees at the apex for patients with differing anatomies. However it is possible that the single blade can be adjusted by changing the angle at the apex 20 which allows the doctor to accommodate patients of different anatomy. The following features are thus provided:

1. A laryngoscope blade is designed such that when it is inserted in the oral cavity to expose the glottis, there exists on the tongue of the blade an inflection point (apex 20) that defines the eyeline deviation angle. The part of the blade between the apex 20 and the larynx can be lengthened or shortened. This allows insertion of the blade to different depths in larger or smaller patients without substantially changing the proportions of the triangle formed by the apex 20 with the line joining the tracheal end of the blade (tip 17) and the point at which the tongue of the blade is crossed by a line between the upper and lower incisor teeth.

2. This blade design allows the forward space (into which the tongue must be displaced) to be maximised for any given eyeline deviation angle regardless of the required depth of insertion.

3. To accommodate a range of patient anatomies, blades of this design can be constructed that differ in the angle or curve at the apex 20. Alternatively, the blade can be made with a pivot at the apex 20 to allow variation in the apex angle.

4. Because the extendable part of the blade is between apex 20 and point T, no thickening is required of any part of the blade that might encroach on the eyeline deviation or the forward space.

5. The blade is mechanically simple and robust. It is easy to clean and sterilise.

6. The blade can be used with available laryngoscope handles, and can be adapted for use with either a bulb or fiber optic light source.

7. The preferred embodiment makes use of a sliding control operated by the free hand to lengthen and shorten the blade, but a lever or knob can also be used. The control can also be arranged for operation by the hand that holds the laryngoscopes handle.

The sequence of steps is as follows:

a) Make an initial adjustment of the length of the blade as estimated by assessing the patient.

b) Insert the blade with the tip 17 in the space between the base of the tongue and the epiglottis, so as to engage the hyoid bone.

c) Lift up on the blade to elevate the epiglottis and allow visualization of the opening to the larynx.

d) If improvement in the position is required, the lifting force is relaxed and the length of the extendable part of the blade is adjusted so as to move the apex 20 to its optimal position. In this position the area into which the tongue must be compressed will be maximized.

e) Lift up again on the blade to visualize the opening to the larynx. The first part (part 13) of the blade will be close to the lower teeth.

f) Repeat steps d) an e) if required.

The blade can be used to lift the epiglottis directly by inserting it a little further before lifting up. (Except for straight blade, this is not generally the preferred method, but it can be used. In some difficult situations a straight blade can be advantageous and in fact the blade can be used in those patients in whom a straight blade might be preferable simply by fully extending the adjustable part when it would otherwise to shorter.)

Since various modifications can be made in my invention as herein above described, and many apparently widely different embodiments of same made within the spirit and scope of the claims without departing from such spirit and scope, it is intended that all matter contained in the accompanying specification shall be interpreted as illustrative only and not in a limiting sense.

1. A laryngoscope comprising:
a bracket for mounting on a handle for manipulation by the doctor;
a blade attached to the bracket for insertion through the mouth of the patient into the throat of the patient;
the blade being generally concave with an inner surface for engaging and compressing the tongue of the patient onto the lower jaw of the patient, a tip portion remote from the bracket for engaging the hyoid bone of the patient to effect moving of the epiglottis and an outer surface over which the doctor obtains a direct line of sight through the mouth past the moved epiglottis to the opening through the larynx to the trachea;
the blade having a thickness between the inner and outer surfaces arranged such that the blade is substantially rigid;
the blade being shaped to define an apex at a position thereon which in use is located adjacent the tongue of the patient;
a portion of the blade from the apex to the tip portion being substantially straight;
a length of the straight portion of the blade from the apex to the tip portion being adjustable.

2. The laryngoscope according to claim 1 wherein the length is adjustable by a manually operable element exposed beyond the mouth of the patient.

3. The laryngoscope according to claim 1 or 2 wherein the length is adjustable by a manually operable push slide.

4. The laryngoscope according to claim 3 wherein the push slide passes through a slide channel in the blade.

5. The laryngoscope according to claim 4 wherein the blade includes a light channel for communicating light to illuminate the area adjacent the tip portion and wherein the slide channel is alongside the light channel.

6. The laryngoscope according to claim 5 wherein the slide channel is between the light channel and the blade.

7. The laryngoscope according to any preceding claim wherein the tip portion is bent at an angle to the straight portion in a direction toward the inner surface.

8. The laryngoscope according to any preceding claim wherein the blade includes a first portion between the apex and the bracket which is arranged at an angle to the straight portion.

9. The laryngoscope according to claim 8 wherein the inner surface of the first portion is straight or has a moderately concave curvature.

10. The laryngoscope according to claim 8 or 9 wherein the first portion has a length which is approximately 40% of the length from the apex to the end of the tip portion in the extended position of the straight portion.

11. The laryngoscope according to any preceding claim wherein the apex has a radius of curvature which is sufficiently short such that the line of sight of the doctor intersects the apex at the same point regardless of movements of the blade in the oral cavity which cause changes of angle of the straight portion.

12. The laryngoscope according to any preceding claim wherein the apex defines an angle in the range 120 to 140 degrees.

13. The laryngoscope according to any preceding claim wherein the blade is generally Z-shaped in cross section forming a raised portion alongside the outer surface for receiving a portion of the tongue of the patient.

14. The laryngoscope according to claim 13 wherein the length is adjustable by a manually operable push slide which passes through a slide channel in the blade at a web in the Z-shape.

15. A method of inserting a laryngoscope into a patient comprising:
providing a laryngoscope according to any preceding claim;
inserting the laryngoscope into the oral cavity of the patient to a position in which the apex is located over the tongue of the patient;
moving the blade to compress the tongue of the patient;
adjusting the position of the apex relative to the tongue to achieve maximum compression;
and with the apex so positioned, adjusting the length of the straight portion to provide contact between the tip portion and the hyoid bone of the patient.
